(19) United States
(12) Patent Application Publication
(10) Pub. No.: US 2023/0270337 A1
(43) Pub. Date: Aug. 31, 2023

(54) SYSTEM AND METHOD FOR VIDEO DETECTION OF BREATHING RATES

(71) Applicant: HB Innovations, Inc., Los Angeles, CA (US)

(72) Inventors: George H. Petkov, Los Angeles, CA (US); Peter Fornell, Los Angeles, CA (US); Borislav Ristic, Los Angeles, CA (US); Ivan Trujillo, Los Angeles, CA (US)

(73) Assignee: HB Innovations, Inc., Los Angeles, CA (US)

(21) Appl. No.: 17/682,645

(22) Filed: Feb. 28, 2022

(51) Int. Cl. A61B 5/00; A61B 5/08

(52) U.S. Cl. CPC A61B 5/0077; A61B 5/0816; A61B 5/746; A61B 5/7257; A61B 2503/045; A61B 2503/06

(57) ABSTRACT

This disclosure generally relates to systems and methods for video detection of breathing rates in a subject, such as a sleeping baby. One or more video cameras may capture video of the subject. The video may be corrected and then a featured signal may be generated from the corrected video. A respiratory rate detector may be built to output a signal that represents breathing of the subject in the video. The signal may be corrected for any double detections. A breathing rate of the subject may be determined, and then the breathing rate of the subject may be output. In some examples, if the breathing rate is determined to be abnormal, an alarm or notification of the abnormality may be generated, or a moving platform on or in which the subject is sleeping may be moved.

### TECHNICAL FIELD

This disclosure generally relates to breathing rate detection in subjects (e.g., adult, child, baby, etc.), and more particularly to a system and method for video detection of breathing rates.

### BACKGROUND

Detecting lack of breathing is critical for babies (e.g., newborn, infant, toddler), as apnea and obstructive apnea are some of the causes of sudden infant death syndrome (SIDS/SUID), especially between 2-6 months. SIDS (or Crib Death) is a leading cause of baby mortality. When an apnea event occurs, the baby may need to be woken up (as the baby may not wake up on its own) to invigorate the brain to start breathing again. Unfortunately, traditional method for detecting lack of breathing in babies (and other subjects) may be deficient.

### SUMMARY

According to one example, a method includes receiving video of a subject, correcting the video, generating a featured signal from the corrected video, and building a respiratory rate detector to output a signal that represents breathing of the subject in the video. The method further includes correcting the signal that represents the breathing of the subject in the video for any double detection, determining a breathing rate of the subject in the video from the corrected signal, and outputting the breathing rate of the subject in the video.

According to another example, a system includes one or more video cameras configured to capture a video of a subject, and a breath rate detection module. The breath rate module is configured to receive the video, correct the video, generate a featured signal from the corrected video, and build a respiratory rate detector to output a signal that represents breathing of the subject in the video. The breath rate module is also configured to correct the signal that represents the breathing of the subject in the video for any double detection, determine a breathing rate of the subject in the video from the corrected signal, and output the breathing rate of the subject in the video.

### BRIEF DESCRIPTION OF THE DRAWINGS

For a more complete understanding of the present disclosure and its features and advantages, reference is now made to the following description, taken in conjunction with the accompanying drawings, in which:

FIG. 1 schematically illustrates one example of a breathing rate detection system.

FIG. 2 illustrates an example method for detecting a breathing rate of a subject.

### DESCRIPTION

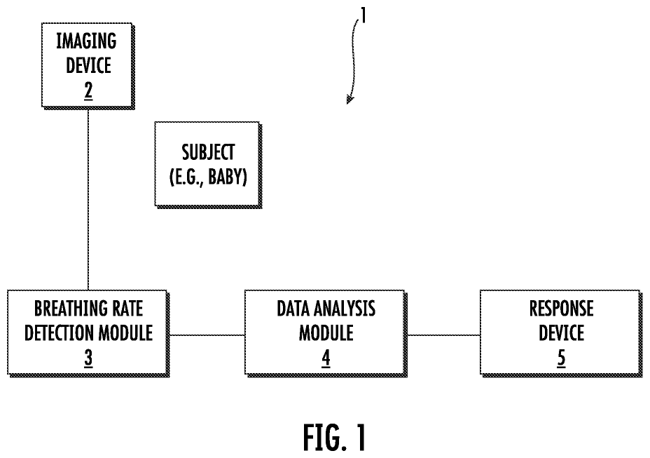

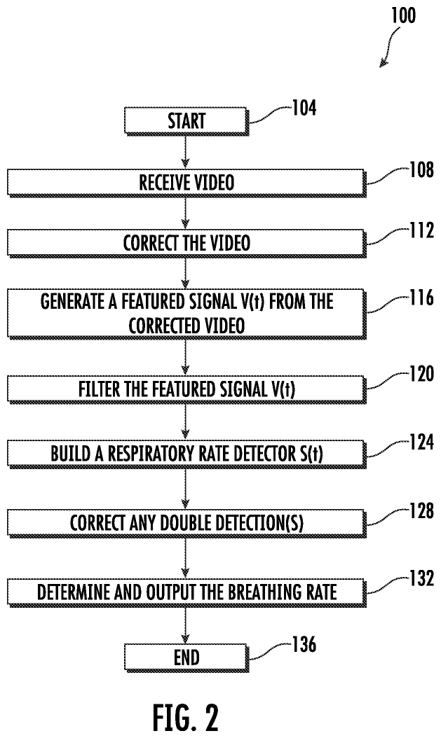

Examples discussed in the present disclosure are best understood by referring to FIGS. 1-2 of the drawings, like numerals being used for like and corresponding parts of the various drawings.

As is discussed above, detecting lack of breathing is critical for babies (e.g., newborn, infant, toddler), as apnea and obstructive apnea are some of the causes of sudden infant death syndrome (SIDS/SUID), especially between the age of 2-6 months. SIDS (or Crib Death) is a leading cause of baby mortality. When an apnea event occurs, the baby may need to be woken up (as the baby may not wake up on its own) to invigorate the brain to start breathing again. Unfortunately, traditional methods for detecting lack of breathing in babies (and other subjects) may be deficient.

The most famous known method for breathing pattern quantification is photoplethysmogram (PPG), which quantifies respiration in a video by using the breathing motions of the respiratory modulation of the PPG. The PPG techniques, however, are not always suitable, as they need skin regions that may not always be visible, and colors fine recognition, which cannot be used at night.

Another known method for breathing pattern quantification involves calculating translation rates with the optical flow, as is described in Koolen N, Decroupet O, Dereymaeker A, Jansen K, Vervisch J, Matic V, et al. Automated Respiration Detection from Neonatal Video Data. Proc 4th Int Conf Pattern Recognit Appl Methods. 2015; 164-9, a copy of which is incorporated herein by reference in its entirety. However, the direct calculation of the translations from the optical flow may contain the projections of many other types of three-dimensional movements into translations in the two-dimensional plane. Hence, this traditional method needs a specific camera position, which may not be conducive to the body position changes that tend to occur during sleep.

A further known method for breathing pattern quantification involves tracking image points over time, as is described in Li MH, Yadollahi A, Taati B. Noncontact Vision-Based Cardiopulmonary Monitoring in Different Sleeping Positions. IEEE J Biomed Heal Informatics. 2017; 21(5):1367-75, a copy of which is incorporated herein by reference in its entirety. However, this method may be problematic because the tracking algorithm tends to refuse to continue tracking a point in some cases, resulting in the loss of tracking points.

An additional known method for breathing pattern quantification involves comparing breathing motion templates, as is described in Wang C W, Hunter A, Gravill N, Matusiewicz S. Unconstrained video monitoring of breathing behavior and application to diagnosis of sleep apnea. IEEE Trans Biomed Eng. 2014; 61(2):396-404, a copy of which is incorporated herein by reference in its entirety. However, this known method may not be feasible, as is needs a colossal template database.

In contrast to this, the systems and methods of FIGS. 1-2 discussed below may address one or more of these deficiencies. For example, the systems and methods of FIGS. 1-2 do not require direct calculation of the translations from the optical flow. As another example, the systems and methods of FIGS. 1-2 do not require tracking and/or templates. In some examples, the systems and methods of FIGS. 1-2 may only track the points in a region of interest by choosing a new set of points during the frame to frame tracking when a loss of tracking points occurs. In some examples, the systems and methods of FIGS. 1-2 may only use templates when a database is already built.

In some examples, the systems and methods of FIGS. 1-2 may detect the breathing rate of a subject by calculating the vector field between a region of interest in two consequent frames, decomposing the movements obtained from an optical flow vector field into six elementary movements in the plane (e.g., two translations, dilatation, rotation, and two shear transformations) to, for example, obtain an approximation of the “pure” movements, excluding the rotation because of its lack of contribution to respiration, choosing a combination of non-overlapped components from the rest of the movement's group, and using these components in a breathing model to extract the breathing rate.

FIG. 1 schematically illustrates one example of a breathing rate detection system 1. The breathing rate detection system 1 may be used to detect the breathing rate of a subject, such as an adult, a child, or a baby. For example, the breathing rate detection system 1 may be used to detect the breathing rate of a subject (e.g., a human baby) that is sleeping (e.g., sleeping in a structure, such as a bassinet). The breathing rate of a subject may refer to the number of breaths taken by the subject during a period of time (e.g., the number of breaths taken during one minute). In some examples, the breathing rate detection system 1 may also (or alternatively) be used to detect respiratory spectral power changes of a subject.

In the illustrated example, the breathing rate detection system 1 includes an imaging device 2. The imaging device 2 may be (or otherwise include) one or more cameras that capture image(s) of a subject, such as one or more images of a human baby sleeping. The camera may be any type of camera, such as a video camera that captures video of the subject. The video refers to sequential images that are ordered to reproduce a visual moving depiction or video of the subject. In the illustrated example, the imaging device 2 is (or otherwise includes) one or more video cameras. The video camera may have a fixed frame rate higher than ten frames per second (fps), in some examples. The video camera may be a standard light video camera (e.g., visible light spectrum), a video camera with low-light capabilities, a video camera with infra-red capabilities, or any combination of the preceding. In some examples, the video camera is a universal serial bus (USB) video camera, a world wide web (WEB) video camera, a video camera that utilizes any other connection format, or any combination of the preceding.

The imaging device 2 may be disposed in an appropriate location relative to and within an appropriate distance from the subject such that it is able to capture images of the subject with sufficient resolution. The images (e.g., video) may be sent to a breath rate detection module 3 by wired (e.g., USB, hardwired, etc.) or wireless electrical signal (e.g., Wi-Fi, Bluetooth, cellular, etc.).

In the illustrated example, the breathing rate detection system 1 further includes the breath rate detection module 3. The breath rate detection module 3 may implement one or more processes to detect the breathing rate of the subject using the data (e.g., video) received from the imaging device 2. Examples of one or more of these processes used to detect the breathing rate are discussed below with regard to FIG. 2. In some examples, to detect the breathing rate, the breath rate detection module 3 may be configured to filter out non-breathing related data from the video received from the imaging device 2, such as non-breathing related motion data. For example, the non-breathing related motion data may arise from the movements of the bassinet, a roll-over movement of the subject, the movement of fabrics around the subject, or any other motion data arising from movement other than the breath of a subject. Examples of this filtering are discussed below with regard to FIG. 2.

The breath rate detection module 3 may include a receiver to receive data (e.g., video) from the imaging device 2. In some examples, the breath rate detection module 3 includes a transmitter or transceiver to transmit signals to the imaging device 2 or to output the determined breathing rate of the subject to a data analysis module 4.

In the illustrated example, the breathing rate detection system 1 includes the data analysis module 4. The data analysis module 4 may be configured to analyze the determined breathing rate of the subject, and may be further configured to make decisions based on the analysis. The data analysis module 4 may be configured to determine if the subject's breathing has stopped by analyzing the determined breathing rate. If there is a loss of the determined breathing rate, the data analysis module 4 may determine if it arises from a breathing stoppage or various non-emergency circumstances, for example, when a baby has been taken out of its crib (or other structure), or there is a physical obstruction of the imaging device 2. Thus, if there is a loss of the determined breathing rate, the data analysis module 4 may be further configured to determine if the loss is abnormal or normal.

The data analysis module 4 may also be configured to output one or more signals based on the analysis performed. For example, the data analysis module 4 may be configured to output a signal to a response device 5, which may be configured to sound an alarm, transmit a call, text, email, or message a caregiver, or control movement of a moving platform of a bassinet, or undertake another action. The data analysis module 4 may output the signal to the response device 5 when abnormal or interrupted baby breathing has been detected. The output signal may be transmitted according to a protocol compatible with a communication protocol of an alert system.

In some examples, the data analysis module 4 may also output statistics regarding the determined breathing rate that may be sent and/or presented to a caregiver and/or medical professional. The statistics may provide information related to sleep parameters and/or patterns, subject (e.g., infant) environment, quality and/or duration of sleep, and/or breathing and/or heartbeat patterns such as frequency and/or variabilities. In some examples, output may include images and/or video recordings of the subject (e.g., infant) that may be sent and/or presented to a caregiver.

In some examples, a subject's breathing profile may be determined over time. An example of processes for determining and utilizing a subject's breathing profile is discussed in U.S. Patent Application Publication No. 2020/0397349, filed Jun. 18, 2020, and entitled “System and Method for Monitoring/Detecting and Responding to Infant Breathing”, a copy of which is incorporated herein by reference in its entirety. The subject's breathing profile may be utilized by the data analysis module 4 to reduce false positives, which are a false diagnosis of abnormal breath stoppage. If the stoppage is determined to be abnormal (e.g., sufficient to reach or exceed a threshold corresponding to a trigger event), the data analysis module 4 may be configured to generate an output signal, as is discussed above.

Although FIG. 1 illustrates the breath rate detection module 3 and the data analysis module 4 as separate modules, in some examples, they may be part of a single combined module. In such an example, the combined module may perform the functions of both the breath rate detection module 3 and the data analysis module 4. For example, the combined module may detect the breathing rate of the subject, analyze the breathing rate of the subject, and transmit an output signal based on the analysis.

In the illustrated example, the breathing rate detection system 1 further includes the response device 5. The response device 5 may be an alert system. The alert system may notify parents, paramedics, or anyone taking care of the subject (e.g., baby), when the output signal is received by the response device 5 from the data analysis module 4. In some examples, the alert system may comprise a cellular, internet, or network server, router, gateway, or other communication device, for example. The alert system may be configured to provide alerts via text message, short message service (SMS), push notification, voice messaging, etc. In some examples, the breathing rate detection system 1 or response device 5 may integrate with and/or communicate with health care/hospital monitoring systems. For example, the system may provide raw or processed data, notifications, and/or alerts to third party systems. The system may also integrate with third party systems. Examples of an alert system are discussed in U.S. Patent Application Publication No. 2020/0397349, filed Jun. 18, 2020, and entitled “System and Method for Monitoring/Detecting and Responding to Infant Breathing”, a copy of which is incorporated herein by reference in its entirety.

The response device 5 may also (or alternatively) be a sound control module, a motion control module, or a communication module. The sound control module, motion control module, or communication control module may be associated with a bassinet. For example, the motion control module may move a platform having a motive platform, for example. Various other signal receiving modules may further be configured to operate the moving platform. Examples of such modules and moveable platforms are discussed in U.S. Patent Application Publication No. 2020/0397349, filed Jun. 18, 2020, and entitled “System and Method for Monitoring/Detecting and Responding to Infant Breathing”, a copy of which is incorporated herein by reference in its entirety.

In some examples, the breath detection system 1 may operate with, be attached to, or be integrated with a bassinet. For example, the breath detection system 1 may be integrated or utilized with a bassinet as disclosed in U.S. Patent Publication No. 2015/0045608, filed Jul. 31, 2014, or U.S. Patent Publication No. 2016/0174728, filed Feb. 26, 2016, or WIPO Publication No. WO 2018/075566, filed Oct. 17, 2017, each of which is hereby incorporated herein by reference in its entirety. In one example, the breath detection system 1 may include a main body comprising a processing module including all or a portion of the breath detection module 3, the data analysis module 4, and/or the response device 5. The main body may be configured to be positioned above the bassinet and roughly centered above an infant laying inside the bassinet. In one example, the main body further comprises the imaging device 2. In another example, a breath detection system 1 may be attached sparsely around a bassinet such that it is able to capture the movement of an infant laying inside the bassinet. In one example, the breath detection system 1 may include a processing module that may be integrated with a bassinet or positioned to receive collected data therefrom, which may include wired or wireless communication with the imaging device 2 comprising one or more video cameras positioned above or around the bassinet. The processing module may be further configured to output a signal, such as a corrective action signal.

The breath detection system 1, the imaging device 2, the breath rate detection module 3, the data analysis module 4, and/or the response device 5 may be embodied in one or more computing systems that may be embedded on one or more integrated circuits or other computing hardware operable to perform the necessary functions of the breath detection system 1, the imaging device 2, the breath rate detection module 3, the data analysis module 4, and/or the response device 5. In some examples, the breath detection system 1, the imaging device 2, the breath rate detection module 3, the data analysis module 4, and/or the response device 5 thereof is integrated with a bassinet having a moving platform. For example, the breath detection system 1, the imaging device 2, the breath rate detection module 3, the data analysis module 4, and/or the response device 5 may be integrated with or be configured for communication with a control system operable to control operations of the moving platform and/or other features of the bassinet. In one example, the breath detection system 1, the imaging device 2, the breath rate detection module 3, the data analysis module 4, and/or the response device 5 comprises a remote device with respect to the bassinet and may communicate with the bassinet or control system thereof via a wireless or wired communication link. In another example, the breath detection system 1, the imaging device 2, the breath rate detection module 3, the data analysis module 4, and/or the response device 5 does not communicate with a control system of the bassinet. In one example, the breath detection system 1, the breath detection system 1, the imaging device 2, the breath rate detection module 3, the data analysis module 4, and/or the response device 5 comprises a portable system allowing a user to position the system with respect to a subject (e.g., baby) to collect data and monitor the same.

FIG. 2 illustrates an example method 100 for detecting a breathing rate of a subject. One or more steps of the method 100 may be performed by the breath detection system 1 of FIG. 1. For example, one or more (e.g., all) of the steps of method 100 may be performed by the breath rate detection module 3 of FIG. 1.

The method 100 begins at step 104. At step 108, video is received. The video refers to sequential images of a subject (e.g., captured by an imaging device 2, such as the video camera, discussed in FIG. 1). For example, the video may be sequential images of a sleeping human baby.

In some examples, the video may be used to detect the breathing rate and/or the respiratory spectral power changes of a subject. When a lack of breathing is detected, the breath detection system 1 of FIG. 1 (or another system executing the method 100) may send an audible signal (e.g., an alarm) to a caretaker to wake up the subject (e.g., baby), or it may send a signal that causes a sleeping device (e.g., a basinet) to wake up the subject (e.g., changing the motion pattern of the baby to wake the baby up). Such signaling may be performed using USB, Wi-Fi, Bluetooth (e.g., Bluetooth low energy (BLE)), the cloud, or other signaling protocols.

In some examples, analysis of the breathing rate may be used to detect other conditions or abnormalities. Example breathing pattern abnormalities may include pneumonia, aspiration, repertory distress syndrome, collapsed lung or any other symptom that cause a deviation in breathing patterns, such as irregular breathing, shallow breathing, rapid breathing and gasping, nasal flaring, grunting, coughing, croup, wheezing, and other symptoms that might be used to inform the parents and the pediatrician of the condition, its severity and duration. The breathing rate analysis may be performed directly on the subject's breath pattern. For example, the baby's breathing pattern may be monitored against itself for developmental progress and abnormal patterns, such as an increase in apnea (duration and frequency). The breathing rate analysis may also (or alternatively) be performed using population data for developmental abnormalities. By collecting breathing patterns from a large number of babies, one can normalize such pattern. By comparing normalized patterns, minor irregularities on a specific subject might be detected. The population data may be analyzed using artificial intelligence (AI) and machine learning (ML) techniques. In some examples, the abnormality detection may be further enhanced using other input modalities, such as sound and vibration sensors placed near and/or underneath the baby.

At step 112, the video is corrected. The video may be corrected in any manner. One example of correcting the video may include determining a region of interest (ROI) in the video. The ROI refers to a portion of the video to concentrate on to detect local movements in the video (e.g., breathing-related movements), as opposed to global movements in the video (e.g., movements of the camera, movements of a device that the baby is lying on, such as the movement of a moving platform on a basinet). The ROI may be determined in any manner. For example, the ROI may be manually determined (e.g., via selection by a user, via defining of the ROI by the user). In other examples, the ROI may be determined using various detection methods, including AI and ML techniques. The ROI used in method 100 for a human subject (e.g., a human baby) is preferably the human's torso (e.g., the baby's torso). In some examples, if a periodical movement (different from breathing caused movements) exists in a portion of the ROI points, one or more of these points may be excluded from the ROI. If a periodical movement (different from breathing caused movements) exists in all of the ROI points, the periodical movement may be removed by stabilizing the video (as is discussed below).

Another example of correcting the video may also (or alternatively) include stabilizing the video. Stabilization of the video may refer to global movement image stabilization of sequential image frames, such as removal of an accumulated global movement to stabilize images while retaining local movement between frames. In some examples, the stabilization may be limited to the ROI while other parts of the video (or individual frames of the video) are ignored. This may be used to more precisely track global movement in a desired region of the image for removal by specifying parts of the frame in which global movement is expected while also reducing computation. For example, the total frame may not contain global movements, but rather only a part of the frame may behave like global movement driven. Example processes for stabilizing a video are described in U.S. patent application Ser. No. 17/136,228, filed Dec. 29, 2020 and entitled “Global Movement Image Stabilization Systems and Methods”, a copy of which is incorporated herein by reference in its entirety.

In some examples, the above described correction of the video at step 112 may be optional. For example, step 112 may include a determination regarding whether a proper ROI has already been determined, and also regarding whether stabilization has already occurred (or whether stabilization is needed). If it is determined that a proper ROI has already been determined, and also if it is determined that stabilization is not needed, the video may be determined to already be in a corrected format. In such an example, the method may move to step 116 (below) without conducting any further correction of the video.

At step 116, a featured signal V(t) is generated from the corrected video. The generation of a featured signal V(t) may include obtaining time series from the corrected video, and then decomposing the time series.

To obtain the time series from the corrected video, the total movements between two consecutive frames of the video may be calculated. That is, the optical flow (OF) between two consecutive frames (which is a velocity vector field) may be calculated. The total movements between two consecutive frames of the video (i.e., optical flow) may be calculated (or otherwise obtained) in any manner. For example, the optical flow may be calculated using a classical Horn-Schunk method, as is described in more detail in Horn B K P, Schunck B G, Determining optical flow, Artif Intell. 1981; 17:185-203, a copy of which is hereby incorporated herein by reference in its entirety. As another example, the optical flow may be calculated using the spectral optical flow algorithm known as SOFIA, as is described in more detail in Kalitzin, S., Geertsema, E. and Petkov, G. 2018, Scale-iterative optical flow reconstruction from multi-channel image sequences, Frontiers in Artificial Intelligence and Applications. 310, (2018), 302-314. DOI: https://doi.org/10.3233/978-1-61499-929-4-302, “Kalitzin et al. 2018a”, a copy of which is hereby incorporated herein by reference in its entirety. As a further example, the optical flow may be calculated using the spectral optical flow algorithm known as GLORIA, which is described in more detail in Kalitzin, S., Geertsema, E. and Petkov, G. 2018, Optical flow group-parameter reconstruction from multi-channel image sequences, Frontiers in Artificial Intelligence and Applications. 310, (2018), 290-301, DOI: https://doi.org/10.3233/978-1-61499-929-4-290, “Kalitzin et al. 2018b”, a copy of which is hereby incorporated herein by reference in its entirety.

The time series may then be decomposed by deriving six time-series from the resulting velocity field of the calculated optical flow. The six time-series may represent the spatial transformations, or group velocities, and include: translation along two image axes (X translation, Y translation), dilation, rotation, shear out, and shear in. These six time-series form a group of motions in the plane V={V_{c}|c=1, . . . , 6}. In some examples, the decomposition may involve decomposing the total vector field using a structural tensor into six elementary movements in the plane, and then finding their main component (movement mean value).

In one example of this, one minute of a movie can be filmed using a camera at 15 frames per second. This results in 900 frames (60 seconds×15 frames/second=900 frames). In such an example the ROI may be 200×300 pixels for a total of 60,000 pixels. The OF over ROI signal, therefore, contains 900 frames (points in time) multiplied by 60,000 vectors. Because a vector in a plane has two numbers, it can be presented by a complex number. Therefore, OF over ROI signal contains 900 time points, and for each time point, it keeps 60 000 complex numbers. In such an example, the featured signal V(t) over ROI contains 900 time points, and for each time point, it keeps six real numbers (e.g., X translation, Y translation, dilation, rotation, shear out, and shear in)

Although the method 100 has been described above as deriving six-time series (i.e., X translation, Y translation, dilation, rotation, shear out, and shear in), in some examples, less than six time-series may be derived, or one or more of the six-time series may be omitted after derivation. As an example of this, when the ROI is in a plane close to the frontal plane, the vectors (elements of the vector field corresponding to the caused by respiration movements) decompose mainly on the axes of dilatation and shear transformations, with only a tiny part of the respiration projected on the axes of translations and eventually rotation. In such an example, the two translations and the rotation vector fields may be omitted for their lack of quantifying the respiratory signal. In other examples, due to different subjects of the video and/or due to different camera positions, only the rotation may be omitted, or a different set of quantifying motions may be selected.

As a result of obtaining the time series from the corrected video, and then decomposing the time series, the featured signal V(t) is generated from the corrected video, in some examples. This featured signal V(t) contains k components V(t)={V_{c}(t)|c=1, . . . , k; k≤6}, in some examples.

At step 120, the featured signal V(t) is filtered. This may, in some examples, allow features of the featured signal V(t) to be analyzed in a specific frequency interval (where the specific frequency interval is, for example, the Frequency of Interest (FOI), the Breathing Frequency of Interest (BFOI), or the interval of frequencies to be used to look for respiratory activity). As one example of this, if a frequency interval [0.5, 1] Hz is desired (e.g., where breathing is defined as a near periodical signal with a frequency between 0.5 and 1 Hz), the featured signal V(t) may be filtered to create a new signal containing only periodicities between 0.5 and 1 Hz. The featured signal V(t) may be filtered in any manner. For example, the featured signal V(t) may be filtered using a band-pass filter (e.g., using Fourier transform, or Gabor wavelets, or a Hilbert-Huang (HH) transform).

In some examples, the filtered signal V(t) is filtered to generate a filtered signal Vh(t). As one example of this, one or more (e.g., all) of the chosen group velocities in the featured signal V(t) is decomposed using the HH transform, with a stop criterion when the residual signal is smaller then δ₂>0. The HH transform refers to a two-step signal processing technique that is applicable to nonstationary and nonlinear signals. In the first step of the HH transform, the input signal is decomposed into different components by using empirical mode decomposition, which are termed as intrinsic mode functions. In the second step of the HH transform, the Hilbert spectrum is obtained by performing the Hilbert transform over the intrinsic mode functions.

Following the decomposition of one or more (e.g., all) of the chosen group velocities in the featured signal V(t) using the HH transform, the respiratory range of frequencies [f₀₁, f₀₂] may then be transformed into oscillations [O₀₁, O₀₂]. Then, a small factor ε>0 may be used to extend the interval [O₀₁, O₀₂] to [O₀₁−ε, O₀₂+ε] for taking into account one or more boundary conditions. A sub-signal Vh(t) may then be formed by summing back all of the HH transform components having oscillations in the interval [O₀₁−ε, O₀₂+ε] for one or more (e.g., all) of the chosen group velocities. This may result in an empirically filtered signal Vh(t) that contains k components Vh(t)={Vh_{c}(t)|c=1, . . . , k; k≤6}.

At step 124, a respiratory rate detector S(t) is built. The respiratory rate detector detects respiration spectral power changes based on a passive remote sensing modality (such as the passive sensing modality provided by a video camera), in some examples. Such spectral power changes may be calculated based on a mean signal over existing channels, in some examples. The respiratory rate detector S(t) outputs a time-dependent signal Sₙ(t) that represents the breathing of the subject, in some examples. For example, the distinct local maxima(s) of the time-dependent signal Sₙ(t) may represent the time(s) of breathing by the subject. In such an example, the distinct local maxima(s) above a certain threshold may represent the times of inhaling and the local minima(s) under a certain point may represent the times of exhaling. The respiratory rate detector S(t) may be built in any manner. Exemplary methods for building the respiratory rate detector S(t) are discussed below.

As a first example, a method for building the respiratory rate detector S(t) may include a vector-field based detector (VFD) method. This VFD method is a data-driven method. In the VFD method, the filtered signal Vh(t) (from step 120) is used directly. That is, the VFD method directly uses the selected components of the movements' vector field between every two consecutive frames (after step 120). The filtered signal Vh(t) represents a series of time vectors, and therefore a complex time series is assumed. In this VFD method, the selected complex time series is summarized (vector sum). Then, the obtained complex time series is divided into time series according to their real and imaginary part(s). The upper spline envelope over the maxima of the real-part time series represents the X coordinates of the proposed respiratory rate detector, corresponding to time. The upper spline envelope over the maxima of the imaginary-part time series represents the Y coordinates of the proposed respiratory rate detector corresponding to magnitude. This results in a determination of VFD(t), which represents the time (i.e., the Xcoordinates) and magnitude (i.e., the Y coordinates) of the proposed respiratory rate detector S(t).

As a second example, a method for building the respiratory rate detector S(t) may include a single-step detector (GSD) method. This GSD method is a model-driven method, and more specifically a detector method that is based on the dimensionality decreasing. In the GSD method, the breath detector may be built from the situational model. The GSD method may be utilized when there is only one type of movement in the frame: the motions caused by the subject's breathing. In such an example, the rest is noise due to the camera itself and the recording conditions. The GSD method may also be utilized when there are several other movements in the frame, partially correlated with breathing, caused by different physical sources, and where there is a linear correlation between the different physical sources and respiration. In such an example, the GSD method eliminates the linear correlations between different data dimensions. In the GSD method, principal component analysis (PCA) may be utilized to eliminate the linear correlations between different data dimensions, so as to erase the noise and release the signal generated by breathing, thereby reducing the dimensions to one. By doing so, the resultant signal (data in principle mode) corresponds to the subject's inhalations and exhalations, corresponding to the local maxima/minima or minima/maxima of the signal, as the principle mode does not preserve the directions on the coordinate axes.

As a third example, a method for building the respiratory rate detector S(t) may include an independent component analysis detector (ICD) method. This ICD method is a model-driven method, and more specifically a detector method that is based on the dimensionality decreasing. The ICD method may be utilized when there are several other movements in the frame, partially correlated with breathing, caused by different physical sources, and a nonlinear correlation or no correlation between the different physical sources and respiration (as opposed to the linear correlation discussed above with regard to the GSD method). In the ICD method, independent component analysis (ICA) may be utilized to eliminate the nonlinear correlations between the signals, resulting in a set of statistically independent signals. This result is based on the idea that different physical sources generate the resulting signals. In some examples, it may not be clear which of the obtained set of signals may represent respiration, and it might also be the case that the breathing signal is not separated as an independent physical source, but different parts of it are involved in the separated signals. In such examples, the ICD method may still be utilized for building the respiratory rate detector S(t), when additional information is available that can answer these questions. Otherwise, it may be advisable to utilize the fourth example (discussed below).

As a fourth example, a method for building the respiratory rate detector S(t) may include a multiple-steps detector (GMD) method. This GMD method is a model-driven method, and more specifically a detector method that is based on the dimensionality decreasing. The GMD method may be utilized when additional information is available that can answer the questions discussed above with regard to the ICD method. For the GMD method, the data is in the principle mode (after, for example, the use of the GSD method, discussed above). In the GMD method, the received signals are embedded in time to obtain the unknown variable and possible time dependencies. Then, the observed time series are converted to state vectors. A phase-space reconstruction procedure may then be used to verify the system order and reconstruct all dynamic system variables while preserving system properties. For each time series, the phase-space dimension and time lag is estimated, and the highest time lag together with its phase-space dimension is chosen. For all the time series obtained so far, the phase space of the uniformly sampled time-domain signal is reconstructed with the chosen couple: a time delay and embedding dimension. Principal component analysis (PCA) may then be used for every newly obtained state vector so as to build a new system (e.g., resulting signals matrix after PCA) with removed time delay correlations (using PCA to put the resulting signals matrix into a principal mode again, removing the linear correlations between the nonlinearities caused by time embedding). In some examples, the resulting signals matrix after PCA may still contain linear or nonlinear trends. In such examples, the typical trending may be removed by summing cumulatively all the time series and de-trending the results. This may result in a system noise that persists in the result, in some examples. If so, the de-trended signal may be decomposed into components using the HH transform, and the first HH transform component may be used as a resulting signal (thereby removing the HH transform residual).

In some examples, the GMD method may not detect the respiratory signal if persistent of another oscillatory signal with a similar or slightly higher power, which masks the respiration. In such example, such signals may be preliminary removed having information about their frequency and amplitudes.

As a fifth example, a method for building the respiratory rate detector S(t) may include an empirical decomposition detector (HFD) method. This HFD method is a model-driven method, and more specifically a spectral contrast based detector. In the HFD method, the empirical decomposition-based detector's signal Sc(t) may be represented by the ratio between: (1) the sum of the second spectrum of the HH transform components of the signals Vh(t) and (2) the sum of the second spectrum of the HH transform component of the signals V(t).

As a sixth example, a method for building the respiratory rate detector S(t) may include a spectral detector (SD) and spectral contrast based detector (SCD) method. This method is a model-driven method, and more specifically a spectral contrast based detector. In the method, to use the frequency (spectral) information, the time-dependent spectral content G(t,v)={G_{c}(t,v)|c=1, . . . , 3} is calculated in each of the chosen group velocities V(t)={V_{c}(t)|c=1, . . . , 3} using convolutions with (for example 200) Gabor filters, using the following equation, with exponentially spaced central frequencies v, in the full range of frequencies [fₛₜₐᵣₜ, f_{end}] Hz, and with α=0.5*v as filter bandwidth.

g(t−t′,v)=(e^{-π}^{²}^{α}^{²}^{v}^{²}^{(t-t′)}^{²}^{-i2πv(t-t′)}−Oᵥ)/Nᵥ (1)

The normalization factor Nᵥ and the offset factor Oᵥ are chosen so that the functions have zero mean and unit 1-norm. To obtain the dominant component of the group velocities Q(t, v), a maximum of the absolute values of the chosen Gabor time-frequency spectra G_{c}(t,v) may be taken, using the following equation:

Q(t,v)=max_{c}(G_{c}(t,v)),c=1, . . . , 3 (2)

where t is time, and c defines the corresponding group velocity

The maximum in the above equation 2 is taken over the group velocities from all cameras (if one has more than one camera, which is preferable).

The Mean Respiratory Power (MRP) is quantified using the following equation:

MRP(t)= [symbol] Q(t,v) [symbol] _{v=f}_{₀₁}^{f}^{₀₂} (3)

The MRP represents a spectral power quantification of the respiratory rate.

In some examples, if MRP is too small, in other words, if using a small, positive threshold δ₁(f₀₁, f₀₂)>0, than MRP<δ₁, it may be the result of two possibilities: (1) the detector in the frequency range of interest contains only low noise and is therefore unreliable; and (2) the strength of the detected signal is comparable to the strength of the spectral background, and their ratio (spectral contrast) represents the detector of interest. As a result, the Relative Respiratory Power (RRP) (see below in equation 4) may be expressed as the ratio of the Gabor power in the respiratory frequency band of [f₀₁, f₀₂] Hz to the total Gabor power in the frequency band of [fₛₜₐᵣₜ, f_{end}] Hz.

$$RRP ( t ) = \{ {\sum }_{v = {f}_{01}}^{{f}_{02}} Q ( t , v ) \} / \{ {\sum }_{v = {f}_{start}}^{{f}_{end}} Q ( t , v ) \} \qquad ( 4 )$$

The breathing rate detector obtained in equation 4 is normalized between zero and one. Therefore, in video segments without movement but breathing motions, the RRP quantity will be close to one, in some examples. In contrast, if no breathing motions are presented, the RRP amount will be close to zero, in some examples. Furthermore, in a relatively constant (stable) background, the RRP quantity will account not for the respiratory rate only, but the respiration power changes as well.

In some examples, no activity may be presented at all, and images without movement may result in an error. To address this, mean total power (MTP) may be introduced as a new quantity, and a small, positive threshold δ₂>0. MTP may be calculated by averaging over all measured frequencies:

MTP(t)= [symbol] Q(t,v) [symbol] _{v=f}_{ₛₜₐᵣₜ}^{f}^{_{end}} (5)

If the MTP. (number of v)<δ₂ and (the nominator of RRP(t))<δ₂ then instead of quantification RRP, MTP may be used. In this example, the resulting quantity will be close to zero, and therefore, no breathing will be detected. The result will contain a warning for a possible empty scene, and no error will appear.

The respiratory rate detector S(t) may be built (in step 124) using two or more of the above discussed exemplary methods. For example, the respiratory rate detector S(t) may be built (by first) utilizing one or more of the first, second, third, and fourth methods (i.e., the VFD method, the GSD method, the ICD method, and the GMD method, discussed above), and then (second) using one or more of the fifth and sixth methods (i.e., the HFD method, and the SD and SCD method, discussed above). Preferably, the respiratory rate detector S(t) may be built (by first) utilizing the fourth method (i.e., the GMD method), and then (second) using the sixth method (i.e., the SD and SCD method). In some examples, more than one of the first, second, third, and fourth methods (discussed above) may be selected for use in building the respiratory rate detector S(t). Furthermore, in some examples, both the fifth and sixth methods may be selected for use in building the respiratory rate detector S(t). The use of additional methods may be used to confirm initial results or for selecting some specificities of the current case. Additionally, to adjust the system's reaction, one or more AI or ML methods may be used for abnormalities detection and recognition.

In some examples, the methods used to build the respiratory rate detector S(t) (in step 124) may be pre-selected. For example, the breath rate detection module 3 may be programed to always utilize the fourth method (i.e., the GMD method), and then the sixth method (i.e., the SD and SCD method). In other examples, the breath rate detection module 3 may dynamically choose which methods to build the respiratory rate detector S(t) based on one or more situations in the video.

At step 128, one or more double detections are corrected in the breathing rate time-dependent signal Sₙ(t), if any exist. A double detection refers to a situation where there are two detections of the same event. The double detection is the result of the camera frame rate being faster than the respiratory rate of the subject, and it is represented in the signal by two consecutive maxima that are too close to each other in time. The double detection may be corrected based on the threshold τ>0. In such an example, if the time difference between two consecutive maxima is smaller than τ (i.e., Δτ_{i,i+1}<τ) the two consecutive maxima may represent two detections of the same event rather than two independent events (with the minima between the two maxima being different noise in the scene). In such an example, the double detection may be corrected by replacing the two maxima (together with the minimum between them) with a single maxima. This replacement may be made using one or more interpolation techniques (such as, for example, linear interpolation) based on one or more assumptions or data observations. One example of the correction of the double detection(s) is included below.

First, using the input signal S(g), δ=τ, the extrema coordinates are calculated: (S, t)={(Sᵢ, tᵢ)|i=1N}.

Second, the total index is set: I={(i)|i=1 . . . N}.

Third, the maxima index is set: K={(m)|m=1 . . . M}.

Fourth, the differences between the maxima's time are determined: Δt={(tₘ₊₁−tₘ)|m=1 . . . M−1}.

Fifth, the following is set: step=0, S(t, step)=S(t)

Sixth, the following is determined: IF min(Δt)<δ. If the answer is no, the new function S(t, step, τ) is returned and the process is ended. Otherwise, if the answer is yes, the process moves to step seven.

Seventh, the following is set: step=step+1

Eighth, the j indices of min(Δt) are found: jₘ, in K; 1≤jₘ≤M−1 & jᵢ, in |, 1≤jᵢ≤N−1

Ninth, new index sets are built: K, | skipping j and j+1 & skipping the corresponding values from the extrema coordinates.

Tenth, the coordinates and the index of the new maxima are computed and added, and the extrema coordinates and indices K, I are rebuilt.

Eleventh, the new function S(t, step) is obtained using cubic spline interpolation over rebuilt extrema coordinates.

Twelfth, the following is determined: IF min(Δt)<δ. If the answer is no, the new function S(t, step, τ) is returned and the process is ended. Otherwise, if the answer is yes, the process re-performs steps seven through twelve until the answer is no. When the answer is no, the new function S(t, step, τ) is returned and the process is ended.

In some examples, correction of double detection(s) in the breathing rate time-dependent signal Sₙ(t) (at step 128) may optionally include building a power spectrum invariant respiratory envelope (threshold α). This power spectrum invariant respiratory envelope may be utilized to correct the breathing rate time-dependent signal Sₙ(t) by removing small maxima that are too close to each other in time (resulting from double detection). Small maxima represents noise in the signal, or some speed changes during respiration. The small maxima refers to a maxima in the signal that is small when compared to the standard deviation of the signal. The small maxima may be removed based on the threshold α>0. This threshold α may be used to remove small, insignificant maxima, resulting from noise or speed change during one respiration cycle. In some examples, the threshold a eliminates a maxima in the signal if the minimum of the absolute values of the differences between its amplitude and the amplitudes of the two surrounding minima is less than α.std(Sₙ(t)).

In some examples, the respiration time-evolution is presented as a sigmoid function, where (1) the maximum speed is in the middle (the centre of the sigmoid); (2) the minimum rate is at sigmoid's both ends; and (3) the sign of the sigmoid may show the stage: inhalation or exhalation. The signal's envelope may highlight or suppress inhalation/exhalation like sigmoid functions with a significant or small difference between the two stages. The terms small/significant depend on the classification threshold α and its relation to the signal's standard deviation. For example:

$$α = \frac{1}{3} ⇒ α . std ( {S}_{n} ( t ) ) = \frac{std ( {S}_{n} ( t ) )}{3} \qquad ( 6 )$$

One example of the building of the power spectrum invariant respiratory envelope (threshold α) is included below.

First, using the input signal S(t, τ), α, the following is calculated: δ=α.std(S(t, τ)).

Second, the extrema coordinates are calculated: (S, t)={(Sᵢ, tᵢ)|i=1N}.

Third, the total index is set: I={(i)|i=1 . . . N}.

Fourth, the following difference is determined: ΔS={abs(Sᵢ₊₁−Sᵢ)|i=1 . . . N−1}.

Fifth, the following is set: step=0, S(t, τ, step)=S(t)

Sixth, the following is determined: IF min(Δt)<δ. If the answer is no, the new function S(t, τ, step, α) is returned, and the process is ended. Otherwise, if the answer is yes, the process moves to step seven.

Seventh, the following is set: step=step+1

Eighth, the j indices of min(Δt) are found: jᵢ, in I; 1≤jᵢ≤N−1

Ninth, a new index is built|skipping j and j+1 & skipping the corresponding coordinate values from the extrema coordinates.

Tenth, the extrema coordinates and index are rebuilt.

Eleventh, the new function S(t, τ, step) is obtained using cubic spline interpolation over new extrema coordinates.

Twelfth, the differences ΔS, and Δt are determined based on the new index.

Thirteenth, the following is determined: IF min(Δt)<δ. If the answer is no, the new function S(t, τ, step, α) is returned, and the process is ended. Otherwise, if the answer is yes, the process re-performs steps seven through thirteen until the answer is no. When the answer is no, the new function S(t, τ, step, α) is returned, and the process is ended.

At step 132, the breathing rate is determined, and then output. The breathing rate may be determined based on distinct local maxima(s) of the time-dependent signal Sₙ(t). For example, the breathing rate may be determined to be the number of positive local maxima above a certain threshold in the time-dependent signal Sₙ(t) output by the respiratory rate detector S(t), divided by the length of the time-dependent signal Sₙ(t) in seconds. This may result in a breathing rate in Hz (which may be changed to breaths per minute by multiplying the result by 60). The breathing rate may be determined for any part/length of the time-dependent signal Sₙ(t) in seconds.

In some examples, determining the breathing rate includes determining respiratory breaths and times. The respiratory breaths correspond to the Y coordinates (local maxima), and the respiratory times correspond to the X coordinates of the time-dependent signal Sₙ(t), in some examples. In some examples, the determined breathing rate also (or alternatively) includes respiratory spectral power changes of a subject.

The determined breathing rate may be used to determine if the subject's breathing has stopped, as is discussed in FIG. 1. The determined breathing rate may be used to determine if the subject's breathing rate is abnormal, such as if the subject has pneumonia or other conditions/symptoms. This determination of abnormality may be case specific, in some examples, and may include a maximum number of seconds without detection of a breath, a non-periodical breath detection (e.g., irregular respiratory intervals), etc.

In some examples, if an issue is determined with regard to the determined breathing rate, an output signal may be transmitted to a response device (e.g., response device 5 of FIG. 1), which may then sound an alarm, transmit a call, text, email, or message a caregiver, or control movement of a moving platform of a bassinet, or undertake another action (as is discussed above with regard to FIG. 1). In other examples, the determined breathing rate may be output in any other manner and to any other device. Examples of the output of the determined breathing rate are discussed above with regard to FIG. 1.

Following step 132, the method 100 moves to step 136, where the method 100 ends.

Modifications, additions, or omissions may be made to method 100. For example, although the steps of method 100 are described above as being performed by breath detection system 1 of FIG. 1 (e.g., performed by the breath rate detection module 3 and/or the data analysis module 4), in some examples, one or more of the steps of method 100 may be performed by any other system, device, and/or module. As another example, one or more steps of method 100 may be optional, or may not be performed. For example, method 100 may not include step 120, where the featured signal V(t) is filtered. As a further example, the steps of method 100 may be performed in parallel or in any suitable order.

Dedicated hardware implementations including, but not limited to, application specific integrated circuits, programmable logic arrays and other hardware devices can likewise be constructed to implement the methods described herein. Applications that may include the apparatus and systems of various examples broadly include a variety of electronic and computer systems. Some examples implement functions in two or more specific interconnected hardware modules or devices with related control and data signals communicated between and through the modules, or as portions of an application-specific integrated circuit. Thus, the example network or system is applicable to software, firmware, and hardware implementations.

In accordance with various examples of the present disclosure, the processes described herein may be intended for operation as software programs running on a computer processor. Furthermore, software implementations can include, but are not limited to, distributed processing or component/object distributed processing, parallel processing, cloud processing, or virtual machine processing that may be constructed to implement the methods described herein. In one example, collected video data of a subject is transmitted directly to a breath detection module comprising a remote data processing resource or may be transmitted to a connection module for transmission to a data processing resource. The data processing resource may comprise a remote processor, which may be distributed, cloud-based, virtual, and/or comprise a remote application or program executable on a server, for example. The video data transmitted may be preprocessed, partially preprocessed, or not processed at all (e.g., raw). A cloud-based service may comprise a public, private, or hybrid cloud processing resource. In an example, the data processing may be performed on the backend of such a system. For example, all or a portion of the breath detection logic may be in the cloud rather than local (e.g., associated with a bassinet or other device in proximity to the baby being monitored). The backend may similarly be configured to generate and/or initiate alerts based on the data processing (e.g., comparison of current breathing to a general or customized breathing profile).

In one example, a breathing detection system, or components thereof, includes a remote resource such as a processor, application, program, or the like configured to receive collected data of the subject. The service may process and analyze the data as described herein (e.g., filter data, generate breathing profiles, modify or update breathing profiles, compare current breathing or breathing patterns to general or customized breathing profiles, determine if current breathing or stoppage is abnormal, communicate and/or integrate with hospital monitoring systems or other third party systems, and/or generate or initiate alerts (e.g., phone call, email, light, sounds, motions, text messages, SMS, or push notifications)). As noted above, the remote resource may comprise a cloud-based service.

The present disclosure describes various modules, which may also be referred to as sub-modules, systems, subsystems, components, units, and the like. Such modules may include functionally related hardware, instructions, firmware, or software. Modules may include physical or logical grouping of functionally related applications, services, resources, assets, systems, programs, databases, or the like. Modules or hardware storing instructions configured to execute functionalities of the modules may be physically located in one or more physical locations. For example, modules may be distributed across one or more networks, systems, devices, or combination thereof. It will be appreciated that the various functionalities of these features may be modular, distributed, and/or integrated over one or more physical devices. It will be appreciated that such logical partitions may not correspond to physical partitions of the data. For example, all or portions of various modules may reside or be distributed among one or more hardware locations.

Various examples described herein may include a machine-readable medium containing instructions such that a system, device, and/or module connected to the communications network, another network, or a combination thereof, can send or receive voice, video or data, and communicate over the communications network, another network, or a combination thereof, using the instructions. The instructions may further be transmitted or received over the communications network, another network, or a combination thereof, via a network interface device. The term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that causes the machine to perform any one or more of the methodologies of the present disclosure. The terms “machine-readable medium,” “machine-readable device,” or “computer-readable device” shall accordingly be taken to include, but not be limited to: memory devices, solid-state memories such as a memory card or other package that houses one or more read-only (non-volatile) memories, random access memories, or other re-writable (volatile) memories; magneto-optical or optical medium such as a disk or tape; or other self-contained information archive or set of archives is considered a distribution medium equivalent to a tangible storage medium. The “machine-readable medium,” “machine-readable device,” or “computer-readable device” may be non-transitory, and, in certain examples, may not include a wave or signal per se. Accordingly, the disclosure is considered to include any one or more of a machine-readable medium or a distribution medium, as listed herein and including art-recognized equivalents and successor media, in which the software implementations herein are stored.

The illustrations of arrangements described herein are intended to provide a general understanding of the structure of various examples, and they are not intended to serve as a complete description of all the elements and features of the systems, devices, modules, and processes that might make use of the structures described herein. Those having skill in the art will appreciate that the breath detection system and processes described herein may find application in many baby apparatuses, such as basinets, bouncy chairs, car seats, or other baby apparatuses in which a baby may sleep and that may include significant non-breathing related motion and/or sounds. Other arrangements may be utilized and derived therefrom, such that structural and logical substitutions and changes may be made without departing from the scope of this disclosure.

Thus, although specific arrangements have been illustrated and described herein, it should be appreciated that any arrangement calculated to achieve the same purpose may be substituted for the specific arrangement shown. This disclosure is intended to cover any and all adaptations or variations of various examples and arrangements of the invention. Combinations of the above arrangements, and other arrangements not specifically described herein, will be apparent to those of skill in the art upon reviewing the above description.

The foregoing is provided for purposes of illustrating, explaining, and describing examples of this invention. Modifications and adaptations to these examples will be apparent to those skilled in the art and may be made without departing from the scope or spirit of this disclosure. Upon reviewing the aforementioned examples, it would be evident to an artisan with ordinary skill in the art that said examples can be modified, reduced, or enhanced without departing from the scope and spirit of the claims described below.

What is claimed is:

1. A method, comprising:
receiving video of a subject;
correcting the video;
generating a featured signal from the corrected video;
building a respiratory rate detector to output a signal that represents breathing of the subject in the video;
correcting the signal that represents the breathing of the subject in the video for any double detection;
determining a breathing rate of the subject in the video from the corrected signal; and
outputting the breathing rate of the subject in the video.

2. The method of claim 1, wherein the subject is a sleeping baby.

3. The method of claim 1, wherein the subject is a sleeping adult or child.

4. The method of claim 1, wherein correcting the signal that represents the breathing of the subject in the video for any double detection comprises replacing two consecutive maxima representing each respective double detection with a single maxima.

5. The method of claim 1, wherein correcting the signal that represents the breathing of the subject in the video for any double detection comprises building a power spectrum invariant respiratory envelope.

6. The method of claim 1, wherein the video of the subject is received from one or more video cameras.

7. The method of claim 1, further comprising utilizing a Fourier transform or Gabor wavelets or a Hilbert-Huang transform to filter the featured signal, wherein the filtered signal is utilized to build the respiratory rate detector to output the signal that represents the breathing of the subject in the video.

8. The method of claim 1, further comprising:
determining that the breathing rate of the subject in the video is abnormal; and
in response to the determined abnormality, causing an alarm or notification of the abnormality to be generated.

9. The method of claim 1, further comprising:
determining that the breathing rate of the subject in the video is abnormal; and
in response to the determined abnormality, causing a moving platform on or in which the subject is sleeping to move.

10. A system, comprising:
one or more video cameras configured to capture a video of a subject; and
a breath rate detection module configured to:
receive the video;
correct the video;
generate a featured signal from the corrected video;
build a respiratory rate detector to output a signal that represents breathing of the subject in the video;
correct the signal that represents the breathing of the subject in the video for any double detection;
determine a breathing rate of the subject in the video from the corrected signal; and
output the breathing rate of the subject in the video.

11. The system of claim 10, wherein the subject is a sleeping baby.

12. The system of claim 10, wherein the subject is a sleeping adult or child.

13. The system of claim 10, wherein, to correct the signal that represents the breathing of the subject in the video for any double detection, the breath rate detection module is further configured to replace two consecutive maxima representing each respective double detection with a single maxima.

14. The method of claim 10, wherein, to correct the signal that represents the breathing of the subject in the video for any double detection, the breath rate detection module is further configured to build a power spectrum invariant respiratory envelope.

15. The system of claim 10, wherein the breath rate detection module is further configured to:
utilize a Fourier transform or Gabor wavelets or a Hilbert-Huang transform to filter the featured signal; and
utilize the filtered signal to build the respiratory rate detector to output the signal that represents the breathing of the subject in the video.

16. The system of claim 10, further comprising a data analysis module configured to:
determine that the breathing rate of the subject in the video is abnormal; and
in response to the determined abnormality, cause an alarm or notification of the abnormality to be generated.

17. The system of claim 10, further comprising a data analysis module configured to:
determine that the breathing rate of the subject in the video is abnormal; and
in response to the determined abnormality, cause a moving platform on or in which the subject is sleeping to move.

18. The system of claim 10, wherein the one or more video cameras and the breath rate detection module are attached to or integrated into a basinet.

19. A method, comprising:
receiving video of a sleeping baby;
correcting the video;
generating a featured signal from the corrected video;
utilizing a Fourier transform or Gabor wavelets or a Hilbert-Huang transform to filter the featured signal;
building a respiratory rate detector to output a signal that represents breathing of the sleeping baby in the video;
correcting the signal that represents the breathing of the sleeping baby in the video for any double detection by replacing two consecutive maxima representing each respective double detection with a single maxima;
determining a breathing rate of the sleeping baby in the video from the corrected signal;
determining that the breathing rate of the sleeping baby in the video is abnormal; and
in response to the determined abnormality, causing an alarm or notification of the abnormality to be generated, or causing a moving platform on or in which the sleeping baby is sleeping to move.
